(19) United States
(12) Patent Application Publication
(10) Pub. No.: US 2008/0299058 A1
(43) Pub. Date: Dec. 4, 2008

(54) Multi-formulation cosmetic compositions

(75) Inventors: Yujin Saito, (US); Hidekazu Tanaka, Toyonaka (JP); Ayumi Horiuchi, Nishinomiya (JP)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company

(21) Appl. No.: 12/156,676

(22) Filed: Jun. 4, 2008

Related U.S. Application Data
(60) Provisional application No. 60/933,075, filed on Jun. 4, 2007.

(51) Int. Cl. A61K 8/18; A61K 31/74

(52) U.S. Cl. 424/59; 424/78.03

(57) ABSTRACT

The present invention relates to a cosmetic composition a) from about 10% to about 90% of water-in-oil emulsion as a first formulation which comprises i) from about 0.1% to about 15% of an emulsifying crosslinked siloxane elastomer; ii) from about 1% to about 40% of a solvent for the emulsifying crosslinked siloxane elastomers; and iii) from about 40% to about 99% of an aqueous phase; and b) from about 10% to about 90% of a second formulation, wherein the first and second formulations are of different formulation, and wherein when shear stress is applied to the composition during spreading on skin, at least a part of the aqueous phase is released from the first formulation.

### CROSS REFERENCE TO RELATED APPLICATIONS

This application claims the benefit of U.S. Provisional Application No. 60/933,075 filed on Jun. 4, 2007.

### FIELD OF THE INVENTION

The present invention relates to multi-formulation cosmetic compositions. Such compositions are useful for delivering skin care actives in products with consumer acceptable sensory and aesthetic benefits, especially as related to smooth spreadability and fresh in-use sensory feeling in particular, for moisturizing, protecting and/or treating the skin and/or keratinous fibers.

### BACKGROUND OF THE INVENTION

Cosmetic products have long been employed to clean and moisturize skin, deliver actives, hide imperfections and protect skin from UV. Cosmetic products have also been used to alter the color and appearance of skin.

To provide such benefits, cosmetic products contain various oily compounds such as emollients and oil-soluble skin care actives which usually accompany unpleasant oily or a tacky feel and poor spreadability. Many cosmetic products also contain particulate materials to provide a unique level of light reflectance or color shift to increase skin radiance, absolve oil, or improve skin cleansing. However, incorporation of certain particulate materials also renders cosmetic products to have a heavy application feel as well as poor spreadability.

Many consumers dislike heavy, oily or greasy feeling compositions and prefer compositions that can provide smooth spreadability and light-feel sensory. Therefore, while delivery of specific skin actives and compounds that can regulate skin conditions is of course important, consumer acceptance of the sensory aspects are also important.

Meanwhile, consumers desiring more benefit and/or protection often will choose a thicker product. For example, products such as a serum, cream or gel composition tends to be perceived as offering greater skin benefits than a clear lotion. Those thicker products, however, if not always, tend to have a resistance to spread which brings poor spreadability.

Cosmetic compositions comprising multiple phases are known in the art field. These products are usually provided in the type of cream, gel, or liquid and are usually focusing on the distinctive appearance and provision of different benefits from each phase. For example, U.S. Pat. No. 4,980,155 to Revlon and U.S. Pat. No. 6,213,166 to Thibiant disclose multiphase compositions which present a unique pattern or shape. U.S. Pat. No. 5,059,414 to Shiseido and WO 2004/26276 to Procter and Gamble disclose two-phase cleansing compositions comprising a cleansing phase and a conditional phase. WO 2006/125598 to Hindustan Lever discloses a multiphase cosmetic composition comprising an emulsion and a gel phase which provides sensory properties by gel phase coming in contact with the skin of the user before the emulsion phase.

Based on the foregoing, there is a need to provide cosmetic compositions with a thickness sufficient to convey an increased benefit, and which provide smooth spreadability to deliver a fresh and pleasant feeling.

None of the existing art provides all of the advantages and benefits of the present invention.

### SUMMARY OF THE INVENTION

The present invention relates to a cosmetic composition comprising a) from about 10% to about 90% of a water-in-oil emulsion as a first formulation which comprises i) from about 0.1% to about 15% of an emulsifying crosslinked siloxane elastomer; ii) from about 1% to about 40% of a solvent for the emulsifying crosslinked siloxane elastomers; and iii) from about 40% to about 99% of an aqueous phase; and b) from about 10% to about 90% of a second formulation, wherein the first and second formulations are of different formulation, and wherein when shear stress is applied to the water-in-oil emulsion, at least a part of the aqueous phase is separated from the water-in-oil emulsion.

The present invention also relates to a cosmetic composition comprising a) from about 10% to about 90% of a water-in-oil emulsion as a first formulation which comprises i) from about 0.1% to about 15% of an emulsifying crosslinked siloxane elastomer; ii) from about 1% to about 40% of a solvent for the emulsifying crosslinked siloxane elastomers; and iii) from about 40% to about 99% of an aqueous phase; and b) from about 10% to about 90% of a second formulation, wherein the first and second formulations are of different formulation, and wherein when shear stress is applied to the composition during spreading on skin, at least a part of the aqueous phase is separated from the water-in-oil emulsion.

The present invention also relates to a method of manufacturing a cosmetic composition of the present invention comprising the steps of a) providing a first formulation and a second formulation separately and b) dispensing the first formulation by a first nozzle and the second formulation by a second nozzle into a container.

The present invention also relates to a method of manufacturing a cosmetic composition of the present invention comprising the steps of a) providing a first formulation and a second formulation separately, b) transferring the first and the second formulations to a vessel, and c) dispensing the first and the second formulations by a nozzle into a container.

The present invention also relates to methods of using such compositions to regulate the condition of mammalian skin. Said methods generally contain the step of topically applying a safe and effective amount of the composition to the skin of a mammal needing such treatment.

These and other features, aspects, and advantages of the present invention will become evident to those skilled in the art from a reading of the present disclosure.

### BRIEF DESCRIPTION OF THE DRAWINGS

FIGS. 1A-B are micrographs of a suitable embodiment of the invention.

FIGS. 2A-C are micrographs of a suitable embodiment of the invention.

FIGS. 3A-C are micrographs of a comparative example.

FIGS. 4-6 are plots of log shear stress (x-axis) versus log viscosity (y-axis) for three suitable embodiments of the invention.

FIG. 7 is a plot of log shear stress (x-axis) versus log viscosity (y-axis) for a comparative example.

FIGS. 8-11 are plots of DAP measurement of suitable embodiments of the invention.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “ambient conditions” as used herein refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein.

As used herein, the “cosmetic product(s)” and “cosmetic composition(s)” are those used to treat or care for, or somehow moisturize, improve, or clean the skin. The “cosmetic product(s)” and “cosmetic composition(s)” include, but are not limited to, moisturizers, personal cleansing products, makeup bases, foundations, occlusive drug delivery patches, nail polish, powders, wipes, hair conditioners, skin treatment emulsions, shaving creams and the like.

The term “keratinous tissue” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “regulating skin condition” as used herein, refers to improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

The term “safe and effective amount” as used herein, refers to an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “thickening agent(s)” as used herein, refers a material increase a viscosity of a composition containing the same.

The term “visibly distinct” as used herein describes compositions in the package or upon being dispensed that display visually different phases. These different phases are either distinctively separate or partially mixed as long as the multiple phase composition remains visible to the naked eye.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

The compositions of the present invention are useful for regulating the skin condition and especially for regulating keratinous tissue condition.

The compositions of the present invention provide additional benefits, including stability, absence of significant (consumer-unacceptable) skin irritation and good aesthetics.

The compositions of the present invention comprise at least two formulations wherein a first formulation of water-in-oil emulsion comprising an emulsifying crosslinked siloxane elastomer, a solvent for the emulsifying crosslinked siloxane elastomers, an aqueous phase, and optionally a non-emulsifying crosslinked siloxane elastomer, and a second formulation which is of different formulation from the first formulation.

The compositions of the present invention also preferably contain one or more skin care actives at least in one formulation. The nature of the actives and other ingredients depending on their nature, can be introduced into an aqueous phase or into an oil phase of the first or the second formulation. The compositions herein may also include a wide variety of other ingredients. The compositions of the present invention are described in detail hereinafter.

In the composition of the present invention, the first and the second formulations may have a viscosity in the range of 30,000-120,000 cps, preferably in the range of 40,000-80,000 cps.

In the composition of the present invention, upon application of shear force or shear stress, phase separation in the first formulation is occurred, and at least a part of an aqueous phase is separated from the first formulation. The phase separation may be visibly observed in some embodiments, while it may not be visibly observed in other embodiments. Without being bound by theory, during application on the skin at least a portion of an aqueous phase of the first formulation may be released from the first formulation, and the released water phase may be present oil layers on the skin and fingers during consumer application, and reducing the friction between the finger and the skin.

In certain embodiments, the release of the aqueous phase may be characterized by the Microscopy Method as presented in the Test Methods. The microscopy method is a microscope-assisted visual analysis of the presence and size of the aqueous domains emulsified within the oil phase. A release of the aqueous phase occurs when an amorphous aqueous region having a maximum linear dimension of at least about 10 microns becomes visible at 500× magnification within about 1 minute of shear. In alternate embodiments, the release of the aqueous phase occurs when an amorphous region of water having a size of at least about 25, 50, or 75 microns becomes visible at 500× magnification within about 1 minute of shear. In another suitable embodiment, the release of the aqueous phase occurs when an amorphous region of water having a size of at least 10 microns becomes visible at 500× magnification within about 45 second, 30 second, or 15 seconds of shear.

In certain embodiments, the release of the aqueous phase may be characterized by phase separation after milling according to the Milling Method provided in the Test Methods. The milling method involves the bulk milling of a 30 g sample of the emulsion. In one embodiment, a release of a portion of the aqueous phase occurs when at least about 0.5 g of the aqueous phase separates after 1 minute of milling at a rate of 24000 rpm. In further embodiments, at least about 1.0 g, 2.5 g, or 5.0 g of the aqueous phase separates after 1 minute of milling at a rate of 24000 rpm. In another embodiment, a release of a portion of the aqueous phase occurs when at least 0.25 g of the aqueous phase separates after 1 minute of milling at a rate of 13500 rpm. In other embodiments, the composition may result in the separation of at least about a 0.5 g portion of the aqueous phase after 1 minute of milling at a rate of 24000 rpm while yielding no release of the aqueous phase (i.e., <0.1 g of aqueous phase) after 1 minute of milling at a rate of 8000 rpm. Examples of shear force or shear stress include applying to the skin or other keratinous tissue, for example by smearing, rubbing, dabbing, wiping, etc. with a finger hand, implement and/or a delivery enhancement device. The released water phase, especially when it is visible, may provide immediately benefits, including but not limited to, an immediate indication that the product is hydrating the keratinous tissue and/or an enhanced pleasant feel upon application.

In certain embodiments, the release of the aqueous phase may be characterized by a viscosity drop as measured in the Rheological Method provided in the Test Methods. The Rheological Method involves applying a controlled stress to a sample of the emulsion to generate a rheology profile of the log of viscosity (y-axis) versus the log of shear stress (x-axis). For an emulsion exhibiting an aqueous phase release upon application of shear, the plot of viscosity versus shear yields a sharp decrease in viscosity at a critical shear stress. The slope of the region of the plot exhibiting a sharp decrease is less than about −5. In alternate embodiments, slope of the region of the plot exhibiting a sharp decrease is less than about −10, −25, −50, −75, or −100.

The composition of the present invention has better spreadability as compared to the second formulation. The spreadability may be measured by the DAP sensory measurement method as present in the Test Methods. The DAP method involves measuring sensory such as Thickness and Rubout Drag measurement performed by trained panels who together function as a calibrated instrument. A higher number in Thickness means a Sample is thicker at 1st rotation to spread the Sample over skin. A higher number in Rubout Drag means a Sample is more resistant to spread over the skin.

### First Formulation

The first formulation of the compositions of the present invention comprises an emulsifying crosslinked siloxane elastomer; a solvent for the emulsifying crosslinked siloxane elastomers; and an aqueous phase. The first formulation is present in the compositions from about 10% to about 90%, preferably from about 30% to about 70%, most preferably from about 40% to about 60% by weight.

### Emulsifying Crosslinked Silicoxane Elastomers

The first formulation of the compositions of the present invention comprises an emulsifying crosslinked siloxane elastomer. The emulsifying crosslinked siloxane elastomer is present in the first formulation of the present invention from about 0.1% to about 15%, preferably from about 0.2% to about 5%, most preferably from about 0.2% to about 2% by weight of the first formulation. The indicated percentages are understood to refer to amount of dry elastomer, as opposed to the total amount of elastomers and solvent, used for example for storage or shipping. The term “emulsifying,” as used herein, means crosslinked organopolysiloxane elastomer having at least one polyoxyalkylene (e.g., polyoxyethylene or polyoxypropylene) or polyglycerin moiety.

Emulsifying crosslinked siloxane elastomers in the present invention include those described in U.S. Pat. Nos. 5,412,002; 5,837,793 and 5,811,487. None-limiting examples of useful emulsifying crosslinked siloxane elastomers are 1) polyoxyalkylene-modified elastomers formed from divinyl compounds, particularly siloxane polymers with at least two free vinyl groups, reacting with Si—H linkages on a polysiloxane backbone. Such emulsifying crosslinked siloxane elastomers are those supplied by Shin-Etsu (KSG-210, KSG-240, KSG-310, KSG-320 and KSG-330). Preferably, the elastomers are dimethyl polysiloxanes crosslinked by Si—H sites on a molecularly spherical MQ resin. Another preferred emulsifying crosslinked siloxane elastomers are siloxane polymers crosslinked with diallyl polyglycerin such as KSG-710 and KSG-810 available from Shin-Etsu.

### Solvent for the Emulsifying Crosslinked Siloxane Elastomer

The first formulation of the compositions of the present invention comprises a solvent for the emulsifying crosslinked siloxane elastomer. Concentrations of the solvent in the first formulation of the compositions of the present invention will vary primarily with the type and amount of solvent and the emulsifying crosslinked siloxane elastomer employed. Concentrations of the solvent may be from about 1% to about 50%, preferably from about 4% to about 40%, more preferably from about 5% to about 30%, by weight of the first formulation.

The solvent, when combined with the emulsifying crosslinked siloxane elastomer particulates, serves to suspend and swell the elastomer particulates to provide an elastic, gel-like network or matrix. The solvent for the emulsifying crosslinked siloxane elastomer is liquid under ambient conditions, and in one embodiment has a low viscosity to provide for improved spreading on the skin.

The solvent for the emulsifying crosslinked siloxane elastomer may comprise one or more liquid carriers suitable for topical application to human skin. These liquid carriers may be organic, silicone-containing or fluorine-containing, volatile or non-volatile, polar or non-polar, provided that the liquid carrier forms a solution or other homogenous liquid or liquid dispersion with the selected crosslinked siloxane elastomer at the selected siloxane elastomer concentration at a temperature of from about 28° C. to about 250° C., preferably from about 28° C. to about 100° C., preferably from about 28° C. to about 78° C. The solvent for the emulsifying crosslinked siloxane elastomer preferably has a solubility parameter of from about 3 to about 13 (cal/cm³)^{0.5}, more preferably from about 5 to about 11 (cal/cm³)^{0.5}, most preferably from about 5 to about 9 (cal/cm³)^{0.5}. Solubility parameters for the liquid carriers or other materials, and means for determining such parameters, are well known in the chemical arts. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988.

The solvent preferably includes volatile, non-polar oils; non-volatile, relatively polar oils; non-volatile, non-polar oils; and non-volatile paraffinic hydrocarbon oils; each discussed more fully hereinafter. The term “non-volatile” as used herein refers to materials that exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm³)^{0.5}.

Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G.E. Silicones, (e.g. SF-1204, SF-1202, GE 7207 and GE 7158); and SWS-03314 (commercially available from SWS Silicones™ Corp.).

Polar, non-volatile oils useful in the present invention include, but are not limited to, silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. In one embodiment, the polar, non-volatile oil is selected from the group consisting of propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); the Dow Corning 200 series (Dow Corning Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.).

Additional solvents useful herein are described in U.S. Pat. No. 5,750,096 to Gerald J. Guskey et al., issued May 12, 1998.

### Aqueous Phase

The first formulation of the compositions of the present invention comprises an aqueous carrier from about 40% to about 99%, preferably from about 50% to about 95%, more preferably from about 65% to about 90%, by weight of the first formulation.

Without being bound by theory, the amount of water phase released from the first formulation and the rate at which water is released from the first formulation can be controlled, depending upon how the oil phase is bonded to the aqueous phase in the first formulation, water-in-oil emulsion. In addition, it is also believed that the amount of the water released from the first formulation and the rate at which it is released from the first formulation can be controlled, for example, by incorporating an additional emulsifier in the aqueous phase and/or the oil phase of the first formulation, by changing the level of the emulsifying crosslinked siloxane elastomer within the claimed range, and by varying the aqueous phase/oil phase ratio.

### Second Formulation

The second formulation is present in the compositions of the present invention at concentrations of from about 10% to about 90%, preferably from about 30% to about 70%, most preferably from about 40% to about 60% by weight. The second formulation of the present invention can be an emulsion or gel.

### Emulsion

Emulsions are heterogeneous systems of liquids such as oil and water and are multiphase systems. In these systems, droplets of one liquid or emulsion are homogenized and stabilized into the other using emulsifiers.

The emulsion phase used as the second formulation can be an emulsion having a continuous aqueous phase such as an oil-in-water and a water-in-oil-in-water emulsion, or an emulsion having a continuous oil phase such as a water-in-oil and oil-in-water-in-oil emulsion. The emulsion for use herein invention comprises 5-70% by weight of an oil, 25-95% by weight of aqueous, and 0.1 to 10% by weigh of an emulsifier.

Suitable oils for the emulsion include, but are not limited to, hydrocarbon oils and waxes, silicone oils, fatty alcohol and fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, salts, isomers and derivatives thereof, and combinations thereof. Non-limiting examples hydrocarbon oils and waxes suitable for use herein include polydecanes, petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof. Non-limiting examples of silicone oils suitable for use herein include dimethicone copolyol, silicone crosspolymers, dimethylpolysiloxane, diethylpolysiloxane, mixed C₁₋₃₀ alkyl polysiloxanes, phenyl dimethicone, dimethiconol, and combinations thereof.

An aqueous phase for the emulsion comprises an aqueous carrier. The aqueous phase may comprise water and/or other hydrophilic substances which exhibit limited solubility in an oil phase, including but not limited to, water-soluble ingredients, water-soluble sunscreens and other water-soluble skin care actives. The aqueous carriers used herein include, but not limited in, water and water solutions of lower alkyl alcohols having 1 to 6 carbons.

A wide variety of emulsifying agents can be employed herein.

In one embodiment, non-limiting examples of which include non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Nonlimiting examples of other emulsifiers for use herein include: polyethylene glycol 20 sorbitan monolaurate (polysorbate 20), steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, Glycereth-25 PCA Isostearate, and mixtures thereof.

In another embodiment, the emulsifier is a silicone emulsifier, including organically modified organopolysiloxanes such as dimethicone copolyols. A wide variety of silicone emulsifiers are useful herein. These silicone emulsifiers are typically organically modified siloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, polyglycerine chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

### Gels

The gel phase used for the present invention can be hydrophilic or hydrophobic in nature, though it is preferably hydrophilic.

A hydrophilic gel is defined as a gel wherein the carrier is hydrophilic. The carrier is preferably water, ethyl alcohol, isopropyl alcohol, or a mixture thereof, more preferably water.

A hydrophobic gel is defined as a gel wherein the carrier is hydrophobic in nature. The carrier is preferably oils, thickened oils, silicone oils, or a mixture thereof.

The gel contains at least one thickening agent as disclosed in the title of Thickening Agents later. The thickening agent is present from about 0.01% to about 2%, preferably from about 0.05% to about 0.5% by weight of the gel.

### Optional Ingredients

In some embodiments, the composition may further comprises at least one compound selected from the group consisting skin care actives, skin conditioning agents, sunscreen agents, particulates, other optional ingredients and mixtures thereof. The compound may be present in either of the first and the second formulations or in both phases.

### Non-Emulsifying Crosslinked Silicoxane Elastomers

The first formulation may further comprise a non-emulsifying crosslinked siloxane elastomer. The term “non-emulsifying,” as used herein, defines crosslinked organopolysiloxane elastomer from which polyoxyalkylene units or polyglycerin units are absent. The non-emulsifying crosslinked siloxane elastomer is present in the first formulation of the composition of the present invention from about 0.1 to about 15%, preferably from about 0.2 to about 5%, most preferably from about 0.2 to about 2% by weight of the first formulation. The indicated percentages are understood to refer to amount of dry elastomer, as opposed to the total amount of elastomers and solvent, used for example for storage or shipping.

Non-limiting examples of non-emulsifying crosslinked siloxane elastomers used herein include dimethicone/vinyl dimethicone crosspolymers, supplied by a variety of suppliers including Dow Corning™ (DC 9040 and DC 9041), General Electric™ (SFE 839), Shin-Etsu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin-Etsu™); hybrid silicone powders comprising a fluoroalkyl group, such as KSP-200 (Shin-Etsu™); and hybrid silicone powders comprising a phenyl group, such as KSP-300 (Shin-Etsu™) and DC-9506 (Dow Corning™).

### Skin Care Actives

The compositions of the present invention may include at least one skin care active. Without being bound by theory, it is believed the present compositions provide versatility in formulating a variety of actives.

In any embodiment of the present invention, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Vitamin B₃ Compounds

Vitamin B₃ compound such as niacinamide is a preferred skin care active for use herein. When used, the vitamin B₃ compound is present invention preferably from about 0.1% to about 30%, more preferably from about 1% to about 20%, even more preferably from about 2% to about 10%.

As used herein, “vitamin B₃ compound” means a compound having the formula:

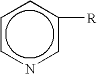

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinoine, kojic acid, arbutin, mulberry extract, undecylenoyl phenylalanine, cetyl pyridinium chloride, glycyrrhizic acid, tetrahydrocurcumin, and mixtures thereof. Use of combinations of whitening agents is also believed to be advantageous in that they may provide whitening benefit through different mechanisms.

When used, the whitening agent is present in the composition from about 0.1% to about 10%, more preferably from about 0.2% to about 5%.

Ascorbic acid compounds are useful whitening agents, and have the formula (I):

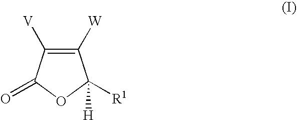

wherein V and W are independently 'OH; R¹ is —CH(OH)—CH₂OH; and salts thereof.

Preferably, the ascorbic acid compound useful herein is an ascorbic acid salt or derivative thereof, such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art.

Undecylenoyl Phenylalanine is the substituted amino acid that is also suitable for use herein as a whitening agent. It is available under the trade name Sepiwhite, from Seppic.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

When included in the present compositions, peptides are preferably included in amounts of from about 1×10⁶% to about 10%, more preferably from about 1×10⁻⁶% to about 0.1%, even more preferably from about 1×10⁻⁵% to about 0.01%, by weight of the composition.

Sugar Amines

The compositions of the present invention may include a safe and effective amount of a sugar amine, which are also known as amino sugars. As used herein, “sugar amine” refers to an amine derivative of a six-carbon sugar.

Examples of sugar amines that are useful herein include glucosamine, N-acetyl glucosamine, mannosamine, N-acetyl mannosamine, galactosamine, N-acetyl galactosamine. Preferred for use herein is glucosamine. Additionally, combinations of two or more sugar amines may be used.

When included in the present compositions, the sugar amine is preferably included in amounts of from about 0.001% to about 20%, more preferably from about 1% to about 10%, even more preferably from about 2% to about 5%, by weight of the composition, of the sugar amine.

### Skin Conditioning Agents

Optionally, the composition of the present invention can further comprise a skin conditioning agent. These agents may be selected from humectants, exfoliants or emollients.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin.

Exfoliants according to the present invention may be selected from C2-C30 alpha-hydroxycarboxylic acids, beta-hydroxycarboxylic acids and salts of these acids. Most preferred are glycolic, lactic and salicylic acids and their ammonium salts.

When the conditioning agent is an emollient it may be selected from hydrocarbons, fatty acids, fatty alcohols and esters.

When used, the amount of skin-condition agent is present in the composition from about 1% to about 60%, preferably from about 2% to about 50%, more preferably from about 5% to about 40%.

### Sunscreen Agents

At least one phase of the compositions of the subject invention may optionally contain a sunscreen agent.

The sunscreen agents are those which generally prevent excessive scaling and texture changes of the stratum corneum by exposure of ultraviolet light. A wide variety of conventional sunscreen agent is suitable for use herein. Preferred are octyl methoxylcinnamate, octyl salicylate, octocrylene, avobenzone, homosalate, octyl triazone, and mixtures thereof. Other conventional Sunscreen agents are also useful herein. Such agents include, for example, butylmethoxydibenzoyl-methane, 2-hydroxy-4-methoxybenzo-phenone, 2-phenylbenzimidazole-5-sulfonic acid, octyldimethyl-p-aminobenzoic acid, 2-ethylhexyl N,N-dimethyl-p-aminobenzoate, p-aminobenzoic acid, oxybenzone, 4-isopropyl dibenzoylmethane, 3-benzylidene camphor, 3-(4-methylbenzylidene)camphor.

Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

When the sunscreen agents are solid, it is preferred to dissolve them in a solvent in view of obtaining higher SPF. Such solvents are generally hydrophobic. Preferred are isopropyl lauroyl sarcosinate, butyloctyl salicylate, diethyl hexyl 2,6-naphthalate, tricaprylin, and mixtures thereof. Solvent useful herein can be also used as the “Emollient” described below.

When included in the present compositions, the sunscreens are present from about 0.1% to about 20%, preferably from about 0.5% to about 10%, more preferably from about 1% to about 5%. Exact amounts will vary depending upon the sunscreen or sunscreens chosen and the desired Sun Protection Factor (SPF).

### Particulates

At least one phase of the compositions of the present invention may optionally contain a particulate.

The particles that can be present in the present invention include inorganic and organic particulates such as talc, mica, sericite, silica, magnesium silicate, synthetic fluorphlogopite, calcium silicate, aluminum silicate, bentonite and montmorillonite; pearl particulates such as alumina, barium sulfate, calcium secondary phosphate, calcium carbonate, titanium oxide, finely divided titanium oxide, zirconium oxide, zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such particulates may be hydrophobically treated or non-hydrophobically treated.

When included in the present compositions, particulates are present in the composition from about 0.01% to about 10%, more preferably from about 0.1% to about 6%, by weight of the composition.

### Thickening Agents

The compositions of the present invention, in some embodiments, may further include one or more thickening agents.

Nonlimiting classes of thickening agents include polymeric thickeners such as carboxylic acid polymers, crosslinked polyacrylate polymers, polyacrylamide polymers, polysaccharides and gums; wax type thickeners such as polyethene, cholesteryl hydorxysterate, beeswax and behenyl alcohol; and metallic soap.

When present, the thickening agent is present in the composition from about 0.01% to about 10%, more preferably from about 0.1% to about 4%.

### Other Optional Ingredients

A variety of additional ingredients can be incorporated into the compositions of the present invention. Nonlimiting examples of these additional ingredients includes; particular materials to modify skin feel or appearance; anti-acne actives; oil-soluble vitamin compounds, terpene alcohols, phytosterol, beta-hydroxy acids such as salicylic acid, and derivatives thereof; chelators; flavonoid compounds; anti-inflammatory agents; anti-cellulite agents; desquamation actives; anti-oxidant/radical scavengers; tanning actives; skin soothing or skin healing actives such as panthenoic acid derivatives (including panthenol, dexpanthenol, ethyl panthenol), aloe vera, allantoin, bisabolol, and dipotassium glycyrrhizinate; antimicrobial or antifungal actives.

### Composition Preparation

The first and second formulations of the present invention are generally prepared by conventional methods such as are known in the art of making topical compositions. Such methods typically involve mixing of the ingredients in one or more steps to a relatively uniform state, with or without heating, cooling, application of vacuum, and the like.

As will be explained in detail in the following context, the composition of each phase is formulated separately. Once formulated, each respective phase can be packed during the packaging process by dispensing the respective formulation or dispensing multi formulations together into a container, such as a jar, pump bottle, tube or the like.

In one preferred embodiment, a method of preparing the composition of the present invention comprises:

a) providing a first formulation and a second formulation separately; and

b) dispensing the first formulation by a first nozzle and the second formulation by a second nozzle into a container

In this embodiment, the first and the second formulations may be dispensed in physical contact each other in the container. In this embodiment, the first formulation may be dispensed into one compartment of a container and the second formulation may be dispensed into the other compartment of the container.

In another preferred embodiment, a method of preparing the composition of the present invention comprises:

(a) providing a first formulation and a second formulation separately;

(b) providing a first formulation and a second formulation separately;

(c) co-dispensing the first and second formulations by a single nozzle into a container.

The compositions of the present invention may be formulated into a facial skin cosmetic, eye cosmetic, lip cosmetic, scalp hair styling aid, facial hair styling aid, moisturizer, wrinkle soothing serum, lotion, mascara, skin facial mask, skin lotion, skin cream, skin gel, eye gel, eye cream, lip gel, lip cream, cosmetic, foundation, or any other commonly known skin product or treatment.

### Product for Topical Use

In one preferred embodiment, the composition of the present invention is a cosmetic composition comprising a water-in-oil emulsion as a first formulation which comprises i) from about 0.1% to about 15% of an emulsifying crosslinked siloxane elastomer; ii) from about 1% to about 40% of a solvent for the emulsifying crosslinked siloxane elastomers; and iii) from about 40% to about 99% of an aqueous phase, and a second formulation which comprises a continuous aqueous phase, and the two formulations are packed in physical contact each other in the same container. In this embodiment, the second formulation may be selected from an oil-in-water emulation, a water-in-oil-in-water emulsion and a hydrophilic carrier based gel, and preferably is an oil-in-water emulation. In this embodiment, the first and second formulations may be coaxially disposed in a container.

In another preferred embodiment, the composition of the present invention is a cosmetic composition comprising a water-in-oil emulsion as a first formulation which comprises from i) from about 0.1% to about 15% of an emulsifying crosslinked siloxane elastomer; ii) from about 1% to about 40% of a solvent for the emulsifying crosslinked siloxane elastomers; and iii) from about 40% to about 99% of an aqueous phase, and a second formulation, and the two phases are packed physically separated each other in a container.

In some of the embodiments, the composition may be packed in a container, such as a jar, pump bottle, squeezable tube or the like.

In some of the embodiments, the first and second formulations may be processed in such a manner that upon dispensing from a container the composition comprises a designated volume ratio of the first formulation and second formulation of the composition.

In some of the embodiments, the first and second formulations in the composition may be visibly distinct.

In some of the embodiments, the second formulation in the composition may further comprise a component selected from the group consisting of skin actives, skin conditioning agents, sunscreen agents, particulates and thickening agents.

### Method of Use

The inventors of the present invention have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appear of spider vessels and varicose veins; 11) methods of masking the appearance of vellus hair on skin; and 12) methods of concealing blemishes and/or imperfections in human skin, including acne, age spots, freckles, moles, scars, under eye circles, birth marks, post-inflammatory hyperparticulateation, etc. Each of the methods discussed herein involve topical application of the claimed compositions to skin.

### Test Methods

### Microscopy Method

This method is a microscope-assisted visual analysis of the presence and size of the water domains within a sample composition (“Sample”). The method uses a standard optical microscope with Differential Interference Contrast and Crossed Polarized Light capabilities and an optical shear stage. Optionally, cross polarization may be used for sample compositions that have low translucency or for characterization of the watery domains. With the cross polarization technique, watery domains will appear dark in the resulting image. A suitable configuration includes a Zeiss Axioplan 2 microscope (available from Carl Zeiss, Inc, Thornwood, N.Y.) coupled with a MTI 3CCD camera (available from DAGE-MTI, Michigan City, Ind.). Images are acquired using Metamorph software version 6.1 (available from Molecular Devices Corporation, Sunnyvale, Calif.) that is used to measure droplet size and save the resulting image. The microscope is paired with a CSS450 optical shear stage (available from Linkam Scientific Instruments, Surrey, UK). The microscope is configured to provide 500× magnification. About 1.5 g of the emulsion (“Sample”) is carefully loaded onto the shear stage to minimize shear. The shear system is configured for a steady mode having a gap width of 1 mm and a constant shear rate of 16 s⁻¹. Temperature is held constant at approximately 25° C. An initial micrograph is captured of the Sample prior to initiation of shear by the shear stage. The sample should have an average water droplet size of about 3 microns or less. If a Sample exhibits an average water droplet size of greater than 3 microns, the Sample may not be properly characterized by microscopy; however, the Sample may be characterized by other methods such as the Milling method. The Sample is subjected to 15 seconds of shear, the shear is discontinued, and a micrograph is captured. This is repeated three times (e.g., Sample is subjected to a cumulative 60 seconds of shear) to yield five micrographs (e.g., taken at time=0, 15, 30, 45, and 60 seconds). The released aqueous phase domains of the Sample are analyzed to provide a maximum linear dimension for each of the released aqueous phase domains. Compositions that do not release aqueous phase when applied to the skin do not exhibit a significant change in the water droplet size when exposed to these conditions.

### Milling Method

This method involves the bulk milling of the sample composition (“Sample”) to provide higher than about 1,000 sec⁻¹ of a shear rate evenly to a Sample, yield a phase separation, and measure weight or amount of the separated phase. The amount of sample and the milling time can be adjusted depending on an equipment type.

As an Example, the milling method involves the bulk milling of a 30 g Sample in 50 mL beaker using an Ultra Turrax T25 mixer with a S 25 KR-18G dispersing element available from IKA Works, Wilmington, N.C. The method is conducted at a temperature of approximately 25° C. The Sample is milled for about 1 minute at a speed of either about 13,500 rpm (which corresponds to a shear rate of about 30,000 s-1) or about 20,500 rpm (which corresponds to a shear rate of about 45,000 s-1). Optionally, a Sample may be milled at a speed of 8,000 rpm (which corresponds to a shear rate of about 17,500 s-1). During the 1 minute of milling, the beaker may be gently (i.e., reciprocating motion of no more than about 1 Hz) moved by hand in a direction parallel to the rotor axis of the mixer. After no more than 5 minutes after milling is ended, phase separation is observed. The aqueous phase is removed from the beaker using standard separation techniques. The separated aqueous phase is weighed.

### Rheological Method

This method provides a rheological profile for a sample composition (“Sample”). The Sample is evaluated using an AR 2000 Rheometer available from TA Instruments, New Castle, Del. that is interfaced with a computer having software that provides data recordation and analysis. The rheometer is configured with 4 cm flat plates at a gap setting of 1000 microns, a temperature of 25° C., and in a controlled stress mode. The rheometer is configured to ramp stress from 1 Pa to 1000 Pa with a duration of 3 minutes and to sample at a rate of 10 points per decade. A rheology profile is plotted using the log₁₀ viscosity (Pa·s) on the y-axis versus the log₁₀ shear stress (Pa) on the x-axis. Water-releasing Samples exhibit a sharp decrease in viscosity at a critical shear stress. This decrease in viscosity may be measured as the slope of the plot between the regions wherein the viscosity has a substantially constant high viscosity and a substantially constant lower viscosity. The slope is calculated according to the formula [(log viscosity(t2)−log viscosity(t1)]/[(log shear stress(t2)−log shear stress (t1)], where viscosity (t1) and viscosity (t2) are the viscosity readings before and after the viscosity value decreases 10 fold (which on the log scale is a change of 1.0) between two readings, and the shear stress (t1) and shear stress (t2) are the corresponding shear stress readings. If the viscosity decreases gradually and no sudden viscosity drop of more than 10 fold between two readings occurs, any representative readings on the plot can be used for the slope calculation.

### Sensory Expert Grading Method (DAP Sensory Measurement)

Blinded, monadic, single-use evaluations are conducted under controlled environmental conditions (20-25° C./45-50% RH), by 10-11 trained expert panels using standardized procedures. Evaluations are done on one side of cheeks. 0.1 ml of a Sample is applied to a washed cheek. Using the pads of the index and middle finger of the dominant hand, the Sample is applied with 15 circular rotations (about 2 inches in diameter) in the center of the cheek. Tactile feel of the Sample is evaluated using the absolute standard score from 0 to 8. Thickness (perception of Sample thickness from point of application over skin surface) and Rubout Drag feel (perception of Sample movement from point of application over skin surface—resistance to movement) of a Sample are evaluated after 1st rotation, and after 3, 10 and 15 rotations, respectively. Higher number in Thickness of a Sample means the Sample is sticker. Higher number is Rubout Drag means the Sample is more resistant to spread over the skin.

Each panelist records its data on a hard-copy pre-coded ballot. Each test site is washed and equilibrated between test Samples.

### Viscosity

A viscosity is measured by a commercially available viscometer like BROOKFIELD DV II+Viscometer with Helipath T-C bar type spindle (BROOKFIELD ENGINEERING LABORATORIES, INC.) at 5 rpm/min at 25° C.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-14

### Water-in-Oil Emulsions for the First Formulation

Water-in-Oil emulsions of Examples 4 and 8-14 were prepared by conventional methods from the following components. Water-in-Oil emulsions of Examples 1-3 and 5-7 are prepared by conventional methods from the following components.

| Examples (values in wt %) | | | | | | | | | | | | | |
|---|---|---|---|---|---|---|---|---|---|---|---|---|---|
| 1 | 2 | 3 | 4 | 5 | 6 | 7 | 8 | 9 | 10 | 11 | 12 | 13 | 14 |

Phase A

| | | | | | | | | | | | | | | |
|---|---|---|---|---|---|---|---|---|---|---|---|---|---|---|
| Dimethicone *1 | 4.0 | 4.0 | 6.0 | 5.0 | 4.0 | 4.0 | 5.0 | 7.5 | 4.0 | 4.09 | 4.0 | 4.0 | 4.0 | 5.0 |
| Polymethyl | 4.0 | 4.0 | 6.0 | — | 4.0 | 4.0 | — | — | — | 4.09 | 4.0 | 4.0 | 4.0 | — |
| silsesquioxane *2 | | | | | | | | | | | | | | |
| DC9040 *3 | 3.0 | 3.0 | 4.5 | — | — | 3.0 | — | — | — | 8.6 | 3.0 | 15.0 | 3.0 | — |
| DC9045 *4 | — | — | — | — | 3.0 | — | — | — | — | — | — | — | — | — |
| KSG-15 *5 | — | — | — | 5.0 | — | — | 2.7 | 2.7 | 2.7 | — | — | — | — | — |
| Cyclopenta- | 3.0 | 3.0 | 6.0 | — | 3.0 | 3.0 | 5.0 | 7.5 | 4.0 | 11.43 | 7.0 | 6.0 | 3.0 | — |
| siloxane *6 | | | | | | | | | | | | | | |
| KSG-210 *7 | 2.5 | 5.0 | 4.0 | 5.0 | 2.75 | 2.75 | 2.3 | 2.3 | 2.3 | 5.37 | 2.75 | 2.75 | 2.75 | 5.0 |
| KF-6028 *8 | — | — | 0.15 | — | — | — | — | — | — | — | — | — | — | — |
| KF-6017 *9 | — | — | — | 0.3 | — | — | — | — | — | — | — | — | — | 0.3 |
| KF-6104 *10 | — | — | — | — | — | — | — | — | 0.5 | — | — | — | — | — |
| Cover Leaf AR- | — | — | 5.0 | — | — | — | — | — | — | — | — | — | — | — |
| 80 5% KF-9901 *11 | | | | | | | | | | | | | | |
| DC-2503 *12 | — | — | — | — | — | — | — | — | — | 7.08 | — | 1.5 | — | — |
| Jeenate 3H *13 | — | — | — | — | — | — | — | — | — | 3.54 | — | — | — | — |
| BX2959S *14 | — | — | — | — | — | — | — | — | — | 0.7 | — | — | — | — |
| Petrolatum | — | — | — | — | — | — | — | — | — | — | — | 0.5 | — | — |
| Cetyl | — | — | — | — | — | — | — | — | — | — | — | 0.5 | — | — |
| Ricinoleate | | | | | | | | | | | | | | |
| SEFA Cottonate *15 | — | — | — | — | — | — | — | — | — | — | — | 0.5 | — | — |
| Fragrance | 0.1 | 0.1 | 0.1 | — | — | — | — | — | — | 0.1 | — | 0.2 | 0.1 | — |

Phase B

| | | | | | | | | | | | | | | |
|---|---|---|---|---|---|---|---|---|---|---|---|---|---|---|
| Glycerin, USP | 10.0 | 10.0 | 30.0 | 10.0 | 7.0 | 10.0 | — | — | — | 10.0 | 10.0 | 10.0 | 10.0 | 10.0 |
| Niacinamide | 5.0 | 5.0 | 5.0 | 5.0 | 5.0 | 5.0 | 4.0 | 4.0 | 4.0 | 5.0 | 5.0 | 5.0 | 5.0 | 5.0 |
| PEG-32 | | | | 2.0 | | | | | | | | | | 2.0 |
| Elestab HP100 *16 | — | — | — | — | 0.1 | 0.1 | — | — | — | — | — | — | — | — |
| Pentylene | 2.0 | 2.0 | 2.0 | 3.0 | — | 3.0 | 2.0 | 2.0 | 2.0 | 3.0 | | 3.0 | 3.0 | 3.0 |
| Glycol | | | | | | | | | | | | | | |
| 1,2-Hexane | — | — | — | — | 3.0 | — | — | — | — | — | 3.0 | — | — | — |
| Diol | | | | | | | | | | | | | | |
| Sodium | 0.5 | 0.5 | 0.5 | 0.5 | 0.5 | 0.5 | 0.5 | 0.5 | 0.5 | 0.5 | 0.5 | 0.5 | 0.5 | 0.5 |
| Chloride | | | | | | | | | | | | | | |
| Panthenol | 0.5 | 0.5 | 0.5 | — | 1.0 | 1.0 | 0.5 | 0.5 | 0.5 | 0.5 | 0.5 | 0.5 | 0.5 | — |
| N-Acetyl | — | 2.0 | — | — | — | — | — | — | — | — | — | — | — | — |
| Glucosamine | | | | | | | | | | | | | | |
| Promatrixyl ® *17 | — | — | 0.353 | — | — | — | — | — | — | — | — | — | — | — |
| Methylparaben | 0.2 | 0.2 | 0.2 | 0.2 | — | 0.2 | 0.2 | 0.2 | 0.2 | 0.2 | 0.2 | 0.2 | 0.2 | 0.2 |
| Sodium Citrate | 0.2 | 0.2 | 0.2 | 0.2 | — | 0.2 | 0.2 | 0.2 | 0.2 | 0.2 | 0.2 | 0.2 | 0.2 | 0.2 |
| Citric Acid | 0.03 | 0.03 | 0.03 | — | — | 0.03 | 0.015 | 0.015 | 0.015 | 0.03 | 0.03 | 0.03 | 0.03 | — |
| Sodium | 0.07 | 0.07 | 0.07 | — | — | 0.07 | 0.07 | 0.07 | 0.07 | 0.07 | 0.05 | 0.05 | 0.07 | — |
| Benzoate | | | | | | | | | | | | | | |
| Ethylparaben | 0.05 | 0.05 | 0.05 | — | — | 0.05 | 0.05 | 0.05 | 0.05 | 0.05 | 0.05 | 0.05 | 0.05 | — |
| Benzyl Alcohol | — | — | — | 0.2 | — | — | — | — | — | — | — | — | — | 0.2 |
| Glydant Plus *18 | — | — | — | — | 0.3 | — | — | — | — | — | — | — | — | — |
| Disodium | — | — | — | 0.1 | — | 0.1 | 0.1 | 0.1 | 0.1 | — | — | — | — | 0.1 |
| EDTA | | | | | | | | | | | | | | |
| Hexamidine | — | — | — | — | — | — | — | — | — | 0.1 | 0.1 | 0.1 | 0.1 | — |
| Diisethanoate | | | | | | | | | | | | | | |
| Kobopearl | — | — | — | — | — | — | — | — | — | — | 2.0 | — | — | — |
| Interfine Blue *19 | | | | | | | | | | | | | | |
| Water | q.s. | q.s. | q.s. to | q.s. | q.s. | q.s. | q.s. | q.s. | q.s. | q.s. to | q.s. | q.s. | q.s. | q.s. |
| | to | to | 100 | to | to | to | to | to | to | 100 | to | to | to | to |
| | 100 | 100 | | 100 | 100 | 100 | 100 | 100 | 100 | | 100 | 100 | 100 | 100 |

*1 E.g., KF96A (6cs). Available from Shin-Etsu, Tokyo, Japan.
*2 E.g., Tospearl 145A, CF 600, or 2000. Available from GE Advanced Materials, Wilton, CT.
*3 12.5% Dimethicone Crosspolymer in Cyclopentasiloxane. Available from Dow Corning, Midland, MI.
*4 12.5% Dimethicone in Cyclopentasiloxane. Available from Dow Corning, Midland, MI.
*5 5% Dimethicone/Vinyl Dimethicone Crosspolymer in Dimethicone. Available from Shin-Etsu, Tokyo, Japan.
*6 E.g., SF-1202 available from GE Advanced Materials, Wilton, CT; SH245 available from Dow Corning, Midland, MI.
*7 25% Dimethicone PEG-10/15 Crosspolymer in Dimethicone. Available from Shin-Etsu, Tokyo, Japan.
*8 PEG-9 Polydimethylsiloxyethyl Dimethicone. Available from Shin-Etsu, Tokyo, Japan.
*9 PEG-10 Dimethicone. Available from Shin-Etsu, Tokyo, Japan.
*10 Polyglyceryl-3 Polydimethylsiloxyethyl Dimethicone. Available from Shin-Etsu, Tokyo, Japan.
*11 Silica, Alumina, Titanium Dioxide, Talc with surface-coat by Dimethicone/Methicone Copolymer. Available in Catalysts & Chemicals Ind. Co. Ltd., Kawasaki, Japan.
*12 Stearyl Dimethicone wax. Available from Dow Corning, Midland, MI.
*13 Polyethylene. Available from Jeen Int'l Corp., Fairfield, NJ.
*14 TiO₂ dispersion available from Kobo Samples, Inc., South Plainfield, NJ.
*15 Available from Procter & Gamble Chemicals, Cincinnati, OH.
*16 Hexamidine Diisethionate. Availabile from Laboratoires Serobiologiques, Paris, France.
*17 0.085% Palmitoyl Pentapeptide-3 in water. Available from Sederma, Edison, NJ.
*18 DMDM Hydantoin, Iodopropynyl butylcarbamate, 1,3 butylenel glycol in water. Available from Lonza Inc., Basel, Switzerland.
*19 Available from Kobo Samples, Inc., South Plainfield, NJ.

In separate suitable containers are added the ingredients of Phase A and Phase B, and each phase is mixed using a suitable mixer (e.g., Anchor blade, propeller blade, IKA T25). When each phase is homogenous, slowly add Phase B to Phase A while mixing Phase A with a suitable mixer (e.g., Anchor blade, propeller blade, IKA T25) until the batch is homogenous.

Selected examples tested according to the Milling Method provide the following water release:

| | Comparative | | | | | |
|---|---|---|---|---|---|---|
| | Example* | Example 4 | Example 7 | Example 8 | Example 9 | Example 14 |
| 8000 rpm | 0.0 g | 0.00 g | 2.35 g | 0.0 g | 0.0 g | 1.38 g |
| 13500 rpm | 0.0 g | 4.66 g | 13.68 g | 5.22 g | 0.43 g | 9.99 g |
| 24000 rpm | 0.0 g | 7.18 g | 18.39 g | 15.05 g | 5.68 g | 10.80 g |

*The Comparative Example is the commercially available Regenerist Daily Regenerating Serum available from The Procter & Gamble Company.

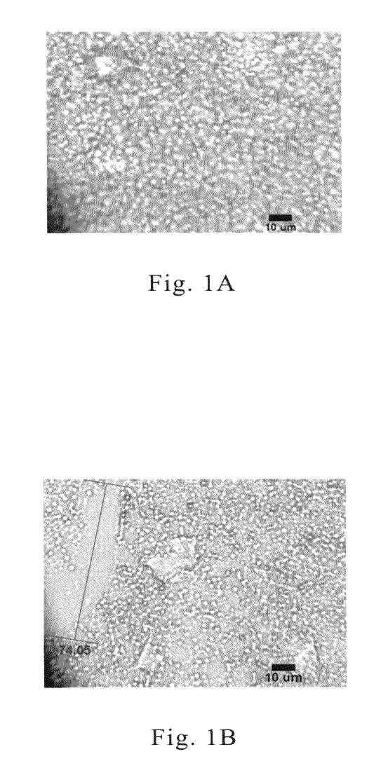

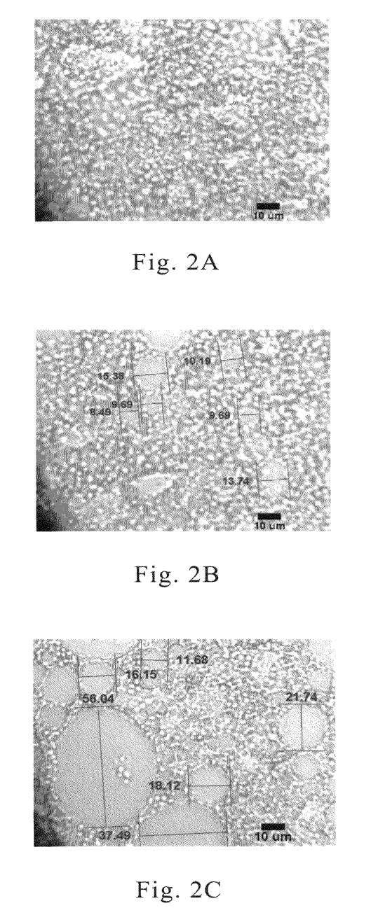

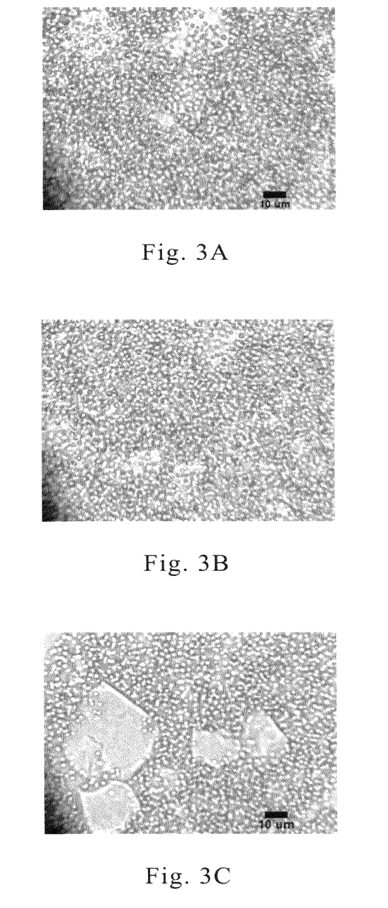

Micrographs for select examples tested according to the microscopy method are provided as FIGS. 1A-B, 2A-C, and 3A-C. The values shown in the micrographs are the approximate longest dimension (in micrometers) of the aqueous domains. FIGS. 1A-B are micrographs of Example 13 taken at 0 seconds and 15 seconds, respectively. FIG. 1B shows an aqueous domain of approximately 74.05 μm after 15 seconds of shear. FIGS. 2A-C are micrographs of Example 12 taken at 0 seconds, 15 seconds, and 60 seconds, respectively. FIG. 2C shows an aqueous domain of approximately 56.04 μm after 60 seconds of shear. FIGS. 3A-C are micrographs of a Comparative Example (commercially available Regenerist Daily Regenerating Serum available from The Procter & Gamble Company) taken at 0 seconds, 15 seconds, and 60 seconds, respectively. FIG. 3C shows silicone elastomer domains that are readily characterized to a skilled microscopist; however, no aqueous domains greater than 10 μm are present.

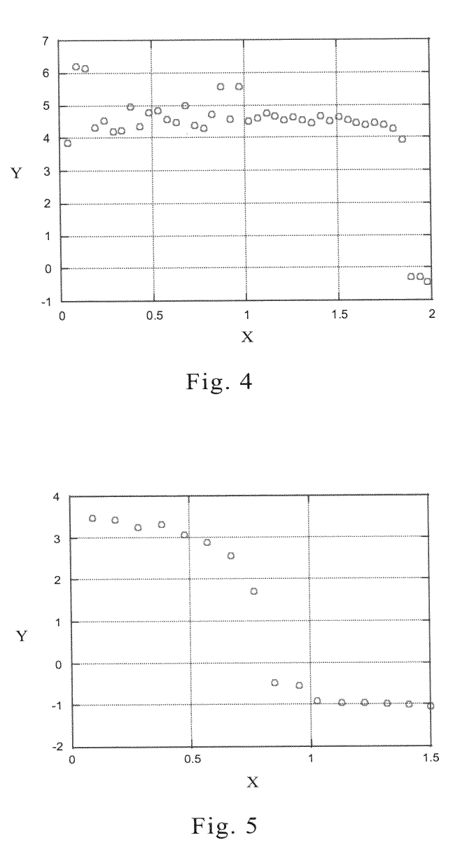

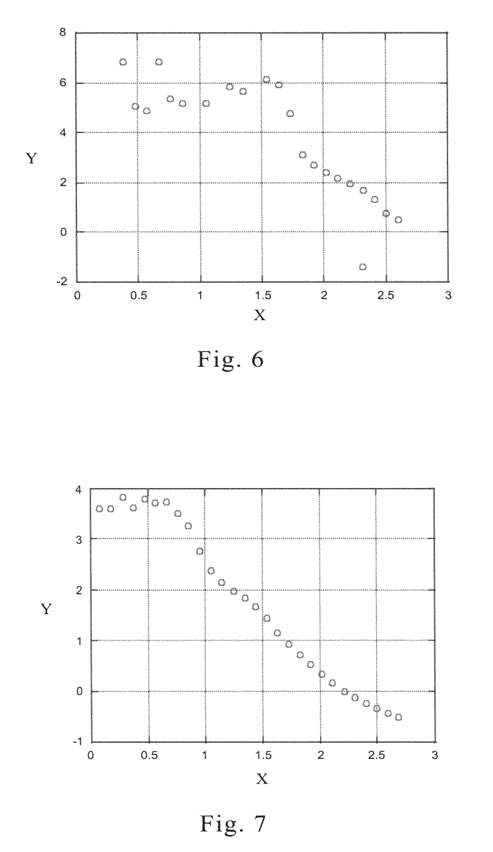

Graphs of the resulting data for select examples tested according to the rheological method are provided in FIGS. 4-7. FIG. 4 is the graph that results from Example 12. FIG. 4 shows a steep drop in viscosity (e.g., slope of about −106) between data points at a shear stress of approximately 1.8 (log). FIG. 5 is the graph that results from Example 11. FIG. 5 shows a drop in viscosity (e.g., slope of about −14.7) between data points at a shear stress of approximately 0.8 (log). FIG. 6 is the graph that results from Example 10. FIG. 6 shows a drop in viscosity (e.g., slope of about −12) between data points at a shear stress of approximately 1.7 (log). FIG. 7 is the graph that results from testing a Comparative Example (commercially available Regenerist Daily Regenerating Serum available from The Procter & Gamble Company). The largest point to point drop in viscosity for the Comparative Example is about −3.4

### Examples 15-22

### Second Formulation

Oil-in-Water emulsions and gels for the second formulation were prepared by conventional methods from the following components.

| | Examples (values in wt %) | | | | | | | |
|---|---|---|---|---|---|---|---|---|
| | 15 | 16 | 17 | 18 | 19 | 20 | 21 | 22 |
| Dimethicone *1 | — | — | — | — | 1.7 | — | — | — |
| Polymethyl | — | — | — | — | 1.5 | — | — | 5 |
| silsesquioxane *2 | | | | | | | | |
| KSG-15 *3 | — | — | — | 2.0 | 2.5 | — | — | — |
| Cyclopenta-siloxane *4 | — | — | — | 8.0 | 8.4 | — | — | — |
| Undecylenoyl | — | — | — | — | 1 | — | — | — |
| Phenylalanine | | | | | | | | |
| Silicone Q2-1503 *5 | 2 | 2 | 2 | — | — | — | — | 2 |
| Isopropyl Isostearate | 1.330 | 1.330 | 1.330 | 2.4 | 2.2 | — | — | — |
| BX2959S *6 | 1.074 | 0.544 | 0.136 | — | 2 | — | — | — |
| SEFA Cottonate *7 | 0.67 | 0.67 | 0.67 | 0.6 | — | — | — | — |
| Glycerin, USP | 6.93 | 6.93 | 10 | 5 | 5 | 4.55 | 5 | 3 |
| Niacinamide | 3.5 | 3.5 | 3.5 | 5 | 5 | — | — | 2 |
| PEG-100 Stearate | 0.1 | 0.1 | 0.1 | — | — | — | — | — |
| Stearyl Alcohol | 0.639 | 0.639 | 0.48 | 0.2 | 1.1 | — | — | 0.6 |
| Cetyl Alcohol | 0.511 | 0.511 | 0.72 | 0.8 | 0.7 | — | — | 0.4 |
| Butylene Glycol | — | — | 2 | — | — | 3 | — | — |
| Behenyl Alcohol | 0.4 | 0.4 | — | — | 0.4 | — | — | — |
| Pentylene Glycol | — | — | — | 3 | 2 | — | 2 | — |
| Methylparaben | 0.1 | 0.1 | — | 0.15 | 0.15 | 0.2 | — | — |
| Boron Nitride SHP-6 | 4 | — | — | — | — | — | — | — |
| Nikkol Nikkomulese LH *8 | — | — | — | 4.5 | 3.5 | — | — | — |
| Keltrol 2% BG-Disp *9 | — | — | — | 5 | — | — | — | — |
| Triethanolamine | — | — | — | | 0.65 | 1.23 | — | 0.55 |
| Silica | 2 | — | — | — | — | — | — | — |
| Pemulen TR-2 2% Disp *10 | — | — | — | — | 6 | — | — | — |
| Triethylhoctanoin | — | — | — | 3.2 | 2.5 | — | — | — |
| Flamenco Satin Orange | 0.5 | — | — | — | — | — | — | — |
| Ethylparaben | 0.05 | 0.05 | 0.2 | — | — | — | — | 0.2 |
| Ethylhexyl | — | — | — | — | — | — | — | 7.5 |
| methoxycinnamate | | | | | | | | |
| Homosalute | — | — | — | — | — | — | — | 5 |
| Octocrylene | — | — | — | — | — | — | — | 5 |
| Steareth-21 | — | — | — | — | — | — | — | 0.9 |
| Methylene bis- | — | — | — | — | — | — | — | 10 |
| benzotriazolyl | | | | | | | | |
| tetranethylbutylphenol | | | | | | | | |
| Phenylbenzimidazole | — | — | — | — | — | — | — | 1 |
| sulfonic acid | | | | | | | | |
| Benzyl Alcohol | 0.25 | 0.25 | 0.25 | 0.2 | 0.2 | — | — | 0.5 |
| Disodium EDTA | 0.1 | 0.1 | 0.1 | 0.1 | 0.1 | — | — | 0.02 |
| Ascorbyl Glucoside | — | — | — | 2 | 2 | — | — | — |
| L-Arginine | — | — | — | 1.02 | 1.02 | — | — | — |
| Carbopol 2% Disp *11 | — | — | — | — | — | 31.25 | 15 | — |
| APS-Gel *12 | — | — | — | — | — | 7 | 35 | — |
| Sepigel 305 *13 | 2 | 2 | 2.25 | — | — | — | — | 3 |
| SK-II Pitera *14 | — | 10 | — | — | — | — | 40 | 5 |
| Water | q.s. to | q.s. to | q.s. to | q.s. to | q.s. to | q.s. to | q.s. to | q.s. to |
| | 100 | 100 | 100 | 100 | 100 | 100 | 100 | 100 |

*1 E.g., KF96A (6cs). Available from Shin-Etsu, Tokyo, Japan.
*2 E.g., Tospearl 145A, CF 600, or 2000. Available from GE Advanced Materials, Wilton, CT.
*3 5% Dimethicone/Vinyl Dimethicone Crosspolymer in Dimethicone. Available from Shin-Etsu, Tokyo, Japan.
*4 E.g., SF-1202 available from GE Advanced Materials, Wilton, CT; SH245 available from Dow Corning, Midland, MI.
*5 88% Dimethicone and 12% Dimethiconol, Available from Dow Corning, Midland MI.
*6 TiO₂ dispersion available from Kobo Products, Inc., South Plainfield, NJ.
*7 Available from Procter & Gamble Chemicals, Cincinnati, OH.
*8 Glycerin and Hydrogenated Lecithin and Hydroxypropyl Methylcellulose Stearoxy Ether and Squalane and Sodium Methyl Stearoyl Taurate, Available from Nikkol Chemicals, Japan.
*9 Water and Butylene Glycol and Xanthan Gum and Methylparaben, Available from Kelco, USA.
*10 Water and Acrylates/C10-30 Alkyl Acrylate Crosspolymer, Available from Nikkol Chemicals, Japan.
*11 Glycerin and Water and Carbomer and Propylene Glycol and Sodium Polyacrylate and Methylparaben and Propylparaben, Available from Noveon, USA.
*12 Glycerin and Water and Carbomer and Propylene Glycol and Sodium Polyacrylate and Methylparaben and Propylparaben, Available from Showa Denko KK, Japan.
*13 40% Polyacrylamide and 30% Water and 24% C13-14 Isoparaffin and 6% Laureth-7, Available from Seppic, France.
*14 Saccharomycopsis Ferment Filtrate and Butylene Glycol and Methylparaben, Available from Procter and Gamble

### Examples 23-28

### Dual Phase Composition

The following cosmetic compositions were prepared by packaging selected first and second formulations into a single container using conventional toothpaste-tube filler equipment.

| | |
|---|---|
| Example 23 | 50% of Example 4 and 50% of Example 15 |
| Example 24 | 50% of Example 4 and 50% of Example 17 |
| Example 25 | 50% of Example 4 and 50% of Example 21 |
| Example 26 | 30% of Example 4 and 70% of Example 21 |
| Example 27 | 10% of Example 4 and 90% of Example 21 |
| Example 28 | 50% of Example 4 and 50% of Example 22 |

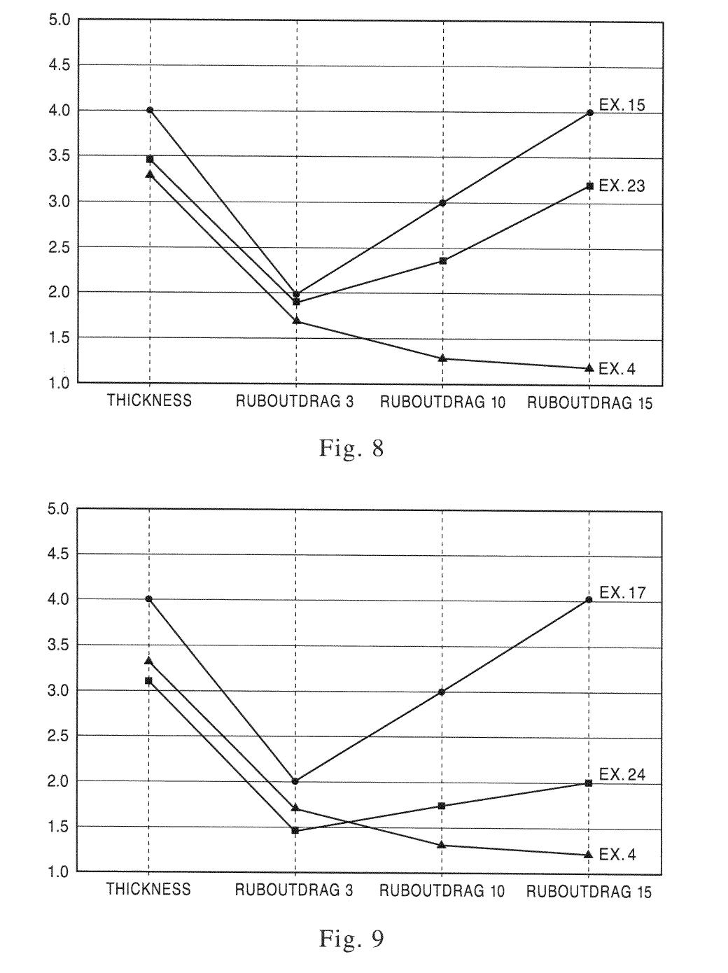

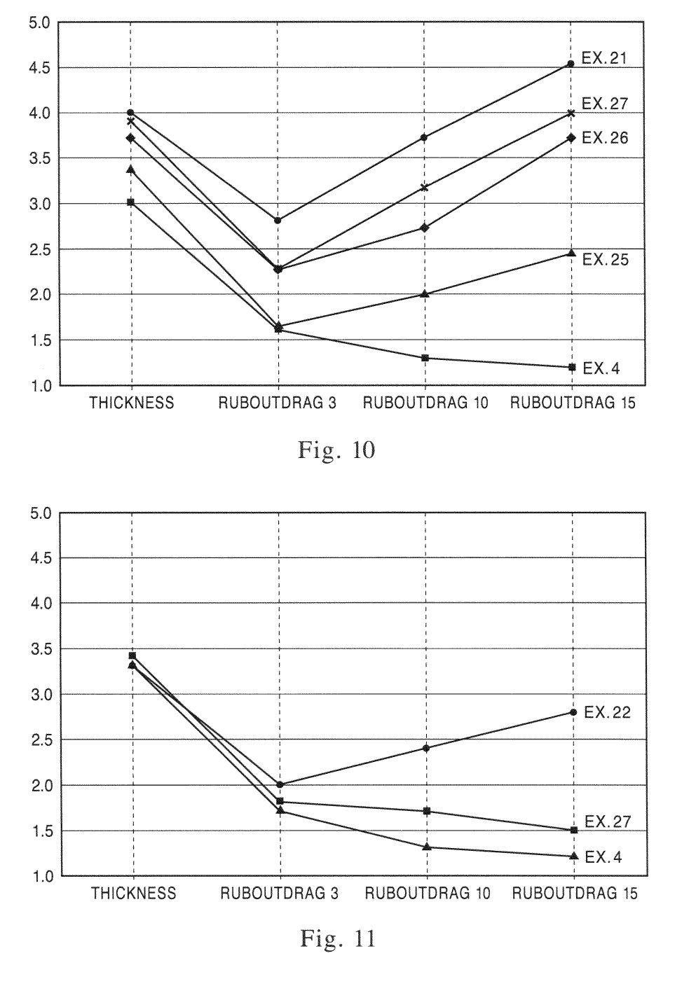

Measurements of Examples 23-28 tested according to the DAP Sensory Measurement method are provided as FIGS. 8-11.

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising:
a) from about 10% to about 90% of water-in-oil emulsion as a first formulation which comprises:
i) from about 0.1% to about 15% of an emulsifying crosslinked siloxane elastomer;
ii) from about 1% to about 50% of a solvent for the emulsifying crosslinked siloxane elastomers; and
iii) from about 40% to about 99% of an aqueous phase; and
b) from about 10% to about 90% of a second formulation;
wherein the first and second formulations are of different formulation; and
wherein when shear stress is applied to the water-in-oil emulsion, at least a part of the aqueous phase is separated from the water-in-oil emulsion.

2. A composition according to claim 1, wherein said composition has better spreadability than the second formulation.

3. A composition according to claim 1, wherein said shear stress is higher than about 1,000 sec⁻¹ of a shear rate.

4. A composition according to claim 1, wherein said first and the second formulations have a viscosity in the range of about 30,000 to about 120,000 cps.

5. A composition according to claim 1, wherein said first formulation further comprises from about 0.1% to about 15% of a non-emulsifying crosslinked siloxane elastomer.

6. A composition according to claim 1, wherein said second formulation is selected from the group consisting of an emulsion and a gel.

7. A composition according to claim 1, wherein said second formulation further comprises a component selected from the group consisting of skin actives, skin conditioning agents, sunscreen agents, particulates and thickening agents.

8. A composition according to claim 1, wherein said first and second formulations are visibly distinct.

9. A composition according to claim 1, wherein said first and second formulations are in physical contact each other.

10. A composition according to claim 9, wherein said first and second formulations are coaxially disposed.

11. A composition according to claim 9, wherein said second formulation comprises a continuous aqueous phase.

12. A composition of claim 1, wherein said first and second formulations are physically separated each other.

13. A cosmetic composition comprising:
a) from about 10% to about 90% of water-in-oil emulsion as a first formulation which comprises:
i) from about 0.1% to about 15% of an emulsifying crosslinked siloxane elastomer;
ii) from about 1% to about 40% of a solvent for the emulsifying crosslinked siloxane elastomers; and
iii) from about 40% to about 99% of an aqueous phase; and
b) from about 10% to about 90% of a second formulation;
wherein the first and second formulations are of different formulation; and
wherein when shear stress is applied to the composition during spreading on skin, at least a part of the aqueous phase is separated from the water-in-oil emulsion.

14. A composition according to claim 13, wherein said first formulation further comprises from about 0.1% to about 15% of a non-emulsifying crosslinked siloxane elastomer.

15. A method of manufacturing the composition of claim 1 comprising the steps of:
a) providing a first formulation and a second formulation separately; and
b) dispensing the first formulation by a first nozzle and the second formulation by a second nozzle into a container.

16. A method of manufacturing the composition of claim 1 comprising the steps of:
a) providing a first formulation and a second formulation separately;
b) transferring the first and the second formulations to a vessel; and
c) dispensing the first and second formulations by a nozzle into a container.

17. A method of regulating the condition of skin comprising applying to the skin of a human in need of treatment, a safe and effective amount of a composition according to claim 1.
